(19) United States
(12) Patent Application Publication
(10) Pub. No.: US 2026/0301926 A1
(43) Pub. Date: Oct. 1, 2026

(54) REMOTE MANAGEMENT PROCEDURE FOR EMERGENCY ROOMS

(71) Applicant: ERAM S.R.L., Empoli (IT)

(72) Inventors: Davide CECIO, Empoli (IT); Leonardo VOLIANI, Empoli (IT); Gianni BIAGI, Empoli (IT); Riccardo SALVADORI, Empoli (IT)

(21) Appl. No.: 19/479,241

(22) PCT Filed: Apr. 26, 2024
(86) PCT No.: PCT/IB2024/054071

§ 371 Date: Oct. 27, 2025

(30) Foreign Application Priority Data
Apr. 27, 2023 (IT) 102023000008313

(51) Int. Cl. G16H 40/20; G16H 40/67

(52) U.S. Cl. CPC G16H 40/20; G16H 40/67

(57) ABSTRACT

A computer-implemented remote management procedure for emergency rooms is envisaged, comprising the following steps: preparing a database including a plurality of priority data, each defining an urgency index; interrogating a plurality of first users via an interface device to generate a plurality of health data, each relating to a respective said first user; recording the health data on the database; analyzing the first health data by executing a first algorithm configured to associate at least one priority data to each of the first health data; organizing said first users based on the priority data so as to classify the first users based on the urgency index defining an ideal ranking; further preparing in the database time data, for each priority data, related to the time of implementation of the priority data; ordering the first users by executing a second algorithm configured to associate the time data to the priority data; and notifying at least one first user associated to a priority data a waiting time, calculated at least by adding the time data of each priority data in the prior ideal ranking to such priority data.

This invention relates to a remote management procedure for emergency rooms of the type specified in the preamble of the first claim. Specifically, this invention pertains to the management of taking charge, sorting, and time control of users who require the intervention of a medical emergency room.

As is known, the emergency room, also denoted by the acronym ER, is an operational unit of the hospital dedicated to emergency or urgency cases. Currently, the emergency room is a place of diagnosis and treatment wherein at least an initial assessment of a user arriving on site is guaranteed.

Normally, a user is evaluated by a professional in charge of reception and is immediately classified for severity or risk of unfavourable evolution, methodically and urgently treated according to the real needs of that precise moment, a diagnosis is made and the user is often discharged after a period of intensive short observation or sub intensive therapy.

In more detail, access is not prioritized based on the order of arrival of people coming into the emergency room, but on the severity of their condition evaluated by the so-called “triage.”

Triage is the first moment of reception and is a function performed by a medical provider, a nurse, specifically trained and having emergency room experience. The nurse, following a methodological, scientific, and dynamic process, identifies care priorities for the users by carrying out a clinical evaluation and the risk of possible unfavourable evolution, and assigning to each user a priority code.

The known art described includes some important drawbacks.

In particular, since triage is carried out on-site, i.e., inside the emergency room hall, overcrowding may occur in particular with a plurality of users having low severity levels, such us for example the green codes, usually assigned to users who experience the longest waiting times, for the obvious reasons since higher severity levels are prioritized.

This entails, in addition to such problems and discomforts inherent to overcrowding for users who may exhibit more or less suffering, also an increase in risks related to diseases or viruses transmissible by air within enclosed spaces.

Thus, the current management of the emergency room, no matter how efficient it may be, entails considerable risks for the users who remain waiting for medical examination inside the emergency room.

In such a situation, the technical task underlying the present invention is to devise a remote management procedure for emergency rooms capable of substantially overcoming at least some of the drawbacks mentioned above.

Within this technical task, an important aim of the invention is to achieve a remote management procedure for emergency rooms that allows reducing the number of users waiting inside the emergency room, especially for those users prioritized with low severity levels.

Another important aim of the invention is to create a remote management procedure for emergency rooms that allows reducing the risk of diseases or viruses transmission within the emergency room, thereby significantly increasing safety within the same.

Furthermore, another task of the invention is to achieve a remote management procedure for emergency rooms that allows optimizing and supporting the work of the healthcare providers working in the emergency room, in particular avoiding overburdening the staff in charge.

The technical task and the specified aims are achieved by a remote management procedure for emergency rooms as claimed in the attached claim 1.

Preferred technical solutions are highlighted in the dependent claims.

In this document, the measures, values, shapes, and geometric references (such as perpendicularity and parallelism), when associated to words like “approximately” or other similar terms such as “almost” or “substantially,” are to be understood as allowing measurement errors or inaccuracies due to production and/or manufacturing errors, and especially allowing a slight deviation from the value, measure, shape, or geometric reference associated to it. For example, such terms, if associated to a value, preferably indicate a deviation not exceeding 10% of that value.

Moreover, when used, terms like “first”, “second”, “upper”, “lower”, “main” and “secondary” do not necessarily identify an order, a priority of relationship or relative position, but may simply be used to more clearly distinguish different components from each other.

Unless otherwise specified, as results from the following description, terms such as “treatment”, “computing”, “determination”, “calculation”, or similar, refer to the action and/or processes of a computer or similar electronic computing device that manipulates and/or transforms data physically represented, such as electronic magnitudes of registers in a computer system and/or memories into, other data similarly represented as physical quantities within computer systems, registers or other devices for storing, transmitting, or displaying information.

Measurements and data reported in this text are to be considered, unless otherwise indicated, as made in International Standard Atmosphere ICAO (ISO 2533:1975).

The remote management procedure for emergency rooms according to the invention is preferably implemented by a computer.

The computer is any processor capable of processing incoming data, preferably digital, in order to generate specific output data.

The remote management procedure broadly includes at least one pre-configuration phase.

In the pre-configuration phase, a database is preferably pre-set. As known, the database is essentially a data bank, i.e., a collection of organized data, which is stored and made electronically accessible.

Thus, the database includes a plurality of priority data.

Each of the priority data defines an urgency index associated to the user health condition. It may identify the triage code.

In other words, the priority data may include information about the urgency of performing a medical intervention depending on health condition and thus in relation to the severity/urgency of the injury and/or problem the user may be suffering of.

The procedure also includes an interrogation phase.

In the interrogation phase, a plurality of first users is interrogated.

The interrogation is carried out, preferably, via an interface device. The interface device may be, for example, a vocal device, configured to emit and capture sounds.

Moreover, it could also include a display and, in addition or alternatively, a keyboard capable of allowing the user to manually enter data.

Moreover, the interrogation can also be carried out, for example, by subjecting each first user to a plurality of questions to which the user must answer in order to generate at least one first health data. For example, the interrogation may consist of a series of consequential questions that the interface device asks to the first user and to which he answers, one at a time; the computer may also be configured to choose, based on the answer, the following question to be asked until it is able to determine with reasonable certainty, and in a precautionary manner, the health condition thus subsequently allowing identifying the level of urgency required by the first user.

In particular, the interrogation allows generating a plurality of first health data. Each first data naturally refers to a respective first user.

The first health data may include a plurality of information related to the health condition of the first user and may substantially represent the updated medical history of the first user in need of emergency care.

Therefore, the first health data is an output value resulting from the interrogation of each first user which is subsequently processed by the procedure.

The procedure also includes a registration phase.

The registration includes recording the first health data on the database.

Thus, during the registration, the first health data collected from the first users in the interrogation phase is grouped within the storage medium whereon the database is defined so that it can be consulted and manipulated from the computer.

Therefore, the procedure includes an analysis phase.

In the analysis phase, the first health data are analysed by executing a first algorithm. The first algorithm is configured to associate at least one priority data with each of the first health data of each respective first user.

Therefore, the algorithm is configured to determine, for each first user, the priority data and thus the respective urgency index.

The first algorithm may, in the simplest embodiment, be configured to read each health data and associate the latter with priority data including, for example, a triage code.

The first algorithm may also be employed to carry out the interrogation.

Therefore, the first algorithm may guide, by formulating sequential questions, the first user to a specific type of intervention so as to allow the definition of the priority data to appropriately correspond to the health data.

However, advantageously, the procedure also includes an organization phase.

In the organization phase, the first users are organized based on the urgency index.

In this way, the first users are classified based on the urgency index so as to generate or define an ideal ranking.

The ideal ranking, therefore, takes into account the needs of each first user by assigning more or less priority depending on the urgency index and then the priority data.

The procedure includes further features.

Preferably, the procedure further includes an additional pre-configuration phase.

In the pre-configuration phase, a time data is prepared on the database. In more detail, on the database there is prepared, for example recorded, a time data for each priority data.

The time data is, therefore, preferably related to the time for carrying out a medical intervention for the priority data. In other words, the time data allows to understand for how long a priority data keeps a space and/or a doctor occupied. For example, the time data defines the time needed to solve each triage code.

The time data may be predefined and/or result from the synthesis of a plurality of historical data.

In this sense, indeed, the procedure may include a configuration of time data. The configuration may include the processing of a plurality of historical data related to each priority data so that the implementation time of a priority data corresponds to the average implementation time of a plurality of priority data having the same urgency index. In other words, the implementation time is given by the average of the actual times acquired based on historical data.

Therefore, the procedure further advantageously includes an ordering phase.

In the ordering phase, the first users are ordered by executing a second algorithm.

The second algorithm is configured, in particular, to associate the time data with the first users in the ideal ranking.

Then, the ideal ranking is enriched with time data that allow the managing of the intervention times for each first user.

The procedure, therefore, advantageously also includes a notification phase.

In the notification phase, a waiting time is notified to a first user being associated to a priority data and thus to an urgency index. The waiting time is calculated essentially by adding the time data of each first user in the prior ideal ranking to the first user to whom the notification is made. Therefore, for example, if a first user appears in the fourth place of the ideal ranking, his waiting time, calculated apart from the interrogation, is assessed by adding the time data relating to the three first users who are before him.

Naturally, the procedure can produce data that is not exclusively achieved from the interrogation of the first users.

In fact, for example, the data base may further include a plurality of second health data. The second health data, if present in the database, is data each one related to a second user.

These data may each be associated to a respective priority data. Such second health data may derive, for example, from manual compilation performed by a nurse at the reception point of an emergency room. Therefore, the second health data do not derive from interrogation but may be preexisting data or otherwise acquired directly in the emergency room and made available to the computer for manipulation together with the first health data.

If the database also includes the second health data, therefore, in the organization phase both the first users and the second users are classified.

The procedure just described essentially processes at least health data of users who approach to at least one emergency room.

However, the procedure may be extended to a plurality of different hospital facilities or even to a plurality of doctors of the same facility who may also be simultaneously engaged.

In this regard, the database may include at least one logistic data.

The logistic data is preferably associated to each priority data. Moreover, the logistic data includes a list of information including availability of reception facilities and/or free doctors.

Advantageously, the logistic data is updated from the computer on the database in real time. This means that, for example, if a doctor or a space of a facility for a priority data are already allocated, the logistic data is updated on the database so as to make the doctor or the facility unavailable.

The procedure may include, therefore, further phases.

For example, the procedure may include a presentation phase.

In the presentation phase, preferably, the list of information is presented to one or more first users. The presentation may be carried out through the interface device.

Moreover, the presentation is carried out preferably at least after the interrogation phase.

Therefore, the procedure may also include a selection phase.

In the selection phase, one or more first users select one among the reception facilities and/or one among the free doctors so that they can be allocated to a reception facility and/or a free doctor. Therefore, the procedure may allow providing a user with data concerning to the availability of facilities or doctors to enable the latter to choose the best option.

Therefore, if the user can choose among a plurality of facilities or doctors, the organization and ordering phases are preferably carried out separately for each reception facility and/or each free doctor.

If the procedure includes the features previously described, it may further include a localization phase.

In this case, the procedure may provide for the localization of a first user and one or more reception facilities through a localization device. The localization device may be a locator of a known type, for example, a GPS locator. Naturally, the localization device could also correspond to or be integrated to the interface device.

For example, the interface device and the locator could be implemented by a smartphone, a tablet, or another electronic device capable of accessing, for example, an online portal managed by the computer and by which the interface with the database can be implemented.

In any case, if there is a localization phase, preferably the procedure includes also a calculation phase. In the calculation phase, a reach time to one or more reception facilities by the first user is calculated. The reach time is assessed based at least on the distance between the first user and the respective reception facility.

Thus, the procedure may include, in the notification phase, the notification of the reach time required by the first user.

The notification may be carried out, for example, before the selection phase. In this way, the first user can obtain an initial overview of the time required to reach the facility as well as, as already described, of the waiting time in the ideal ranking of the facility.

Naturally, the classification of users can be advantageously manipulated in real time so as to be able to manage an increasing number of users.

In particular, in this regard, preferably the procedure includes an updating phase.

In the updating phase, the ideal ranking is updated at least whenever the first and/or second health data of a first user and/or a second user is registered.

Therefore, the procedure could provide a new notification phase to one or more first users whenever an update of the database and, therefore, of the ideal ranking is carried out.

Moreover, the completed ideal ranking, for example, over a specific period of time such as an entire working day, can be used to assess the performance of the procedure.

Indeed, the latter may include an acquisition phase wherein at least a real ranking of a plurality of users related to at least a predetermined quantity of priority data is acquired.

The real ranking may be, for example, entered into the database through manual compilation by an operator in possession of a daily report about for example the activity, of an emergency room.

Therefore, the procedure may include a comparison phase.

In the comparison phase, the real ranking is compared with the ideal ranking related to the same quantity of users. In this way, it is possible to detect any discrepancies between the time data of the ideal ranking and the time data of the real ranking.

Therefore, the procedure may include a correction phase.

In the correction phase, it may be considered to correct the time data in the database so as eliminate the discrepancies.

In other words, the purpose of the correction is to bring ideal time data closer to realistic time data so as to allow notifying to users reliable and punctual waiting times.

The invention enables to implement a remote management system for emergency rooms.

The system essentially comprises, means to implement a procedure as described above.

Moreover, the invention enables the implementation of a particular computer program. The latter includes instructions that, when the program is executed by the computer, allow the computer to carry out a procedure as described above.

Moreover, the program can be transmitted by a signal (for example, electrical or optical) and for example conveyed by, an optical cable or a radio signal. It can be downloaded from a network such as the Internet.

The program can implement, in detail, self-learning mechanisms, for example, machine learning techniques of the “Supervised Learning” type.

A supervised learning algorithm takes a known set of input data, for example, data from the ideal rankings, and known answers to the data defining the outputs, for example, given by the real rankings, and trains a model to generate reasonable predictions for the answer to new data. Therefore, the program can use classification and regression techniques to develop predictive models.

Therefore, the invention enables the implementation of a particular storage support.

The latter is preferably readable by a computer and includes instructions that, when executed by the computer, allow the computer to carry out a procedure as described above.

Moreover, according to an embodiment, the proposed technique is implemented by software and/or hardware modules. In this document, the term “module” corresponds to a software and/or hardware component or a set of hardware and software components.

A software component identifies at least a computer program, one or more subprograms or program libraries, and generally any element of a program or piece of software capable of implementing a function or a set of functions as described in this document. The software module is executed by a data processor of a physical entity (terminal, server, gateway, router, etc.) and is capable of accessing the hardware resources of that physical entity (memories, recording media, communication buses, electronic input/output cards, user interfaces, etc.).

A hardware component corresponds to any element or hardware assembly capable of implementing a function or a set of functions as described below for the component in question. It can be a programmable hardware component or a component with an integrated processor for executing software, for example, an integrated circuit, a smart card, a memory card, an electronic card for executing firmware, etc. Each component of the system described above can obviously implement its own software components. The different embodiment forms mentioned above can be combined with each other to implement the proposed technique.

The remote management procedure for emergency rooms according to the invention achieves important advantages.

Indeed, the remote management procedure for emergency rooms allows managing possible queues by detecting the level of urgency and thus reducing the number of users waiting inside the premises of the emergency room, especially if the users are prioritized with lower severity levels.

In particular, indeed, the procedure allows notifying a reasonable waiting time for the user who, with this information, can avoid crowding the reception facility and can go to the site only at the time indicated by the interface device.

Naturally, the notification can be carried out again following the update of the ideal ranking by the system so as to keep the user always updated on the times to be respected.

Moreover, the remote management procedure for emergency rooms allows reducing the risk of diseases or viruses transmission within the emergency room.

Indeed, avoiding the crowding by waiting users in such contexts reduces the possibility of contaminations.

Therefore, the procedure certainly allows to significantly increase the safety within such premises.

In conclusion, the remote management procedure for emergency rooms allows optimizing and supporting the work the healthcare providers present in the emergency room, particularly by avoiding the overburdening of the staff in charge.

Indeed, the management of the reception will no longer need to be carried out by qualified personnel being, therefore, employed for other tasks or functions.

The invention is susceptible to variations within the scope of the inventive concept defined by the claims.

For example, the procedure may further include a test phase.

The test phase is preferably subsequent to the analysis phase. Moreover, in the test phase, preferably, at least one urgency index determined for a first user is compared with a real urgency index diagnosed, for example, by medical personnel.

Thus, if the determined urgency index corresponds to the real urgency index, the test phase ends positively. Alternatively, if the test phase has a negative outcome, because the determined urgency index and the real one do not match, the procedure includes a revision phase appropriately following at least one negative test phase, i.e., with a negative outcome.

In the revision phase, preferably, the procedure executes a third algorithm configured to retrace the questions asked to the first user and identify the question, preferably the first question identified by going backwards, that led to the mismatch between the determined urgency index and the real index. In other words, the third algorithm retraces the path of questions made during the interrogation phase; the question identified in this revision phase will be the one from which, by varying the answer given by the first user, the real urgency index could be obtained and in particular, a sub-sequence of questions leading to the real urgency index.

The revision phase may be implemented on a plurality of first users interrogated and whose test phase had a negative outcome, so as to be able to identify with greater statistical frequency the question or the questions that most frequently can give rise to negative test phases.

Therefore, the third algorithm may also be configured, in the revision phase, to rework, i.e., modify the sequence of questions to be submitted to the first user so as to reduce or completely eliminate the possibility of running into negative test phases.

For example, the identified question may then be asked again at the end of a triage, i.e., as the last question in the interrogation phase, optionally with the same sequence of answers by the user.

Said identified question may be asked again with the same answer options that the same question had in the past; alternatively, the identified question may be then asked again with the option previously chosen by the first user and the one that gives (or at least could lead to) the aforementioned real urgency index.

In this context, all details are replaceable by equivalent elements and materials, shapes, and sizes can be any.

1. Computer implemented remote management procedure for emergency rooms characterized by comprising:
preparing a database including a plurality of priority data each defining an urgency index;
interrogating a plurality of first users via an interface device to generate a plurality of health data each relating to a respective said first user;
recording said health data on said database;
analyzing said first health data by executing a first algorithm configured to associate at least one said priority data with each of said first health data of each respective said first user so as to determine, for each said first user, said urgency index and therefore said priority data;
an organization phase wherein said first users are organized based on said priority data so as to classify said first users based on said urgency index defining an ideal ranking;
further preparing in said database a time data, for each said priority data, regarding time of implementation of said priority data;
ordering said first users by executing a second algorithm configured to associate said time data to said first users organized in said ideal ranking; and
notifying, at least one said first user associated to a said priority data, a waiting, time calculated at least by adding said time data of each of said first users in said previous ideal ranking to said first user.

2. Procedure according to claim 1, wherein said database includes a plurality of second health data each relating to a second user and each already associated to a respective priority data and wherein, in said organization phase, both said first users and said second users are classified.

3. Procedure according to claim 1, wherein said database includes at least one logistic data updated in real time, associated to each said priority data and including a list of information comprising the availability of reception facilities and/or free doctors, and said procedure comprises:
presenting said list of information to one or more of said first users, and
a selection wherein said one or more first users select one among said reception facilities and/or one among said free doctors so that they can be allocated in a said reception facility and/or to a said free doctor,
said organization and ordering phases being carried out separately for each of said reception facilities and/or each of said free doctors.

4. Procedure according to claim 3, comprising:
locating said first user and one or more of said reception facilities via a localization device, and
calculating a reach time of one or more of said reception facilities by said first user based at least on the distance therefrom;
notifying said reach time to said first user.

5. Procedure according to claim 1, comprising configuring said time data by processing a plurality of historical data relating to each said priority data so that said implementation time of a said priority data corresponds to the average implementation time of a plurality of said priority data having said same urgency index.

6. Procedure according to claim 1, comprising updating said ideal ranking at least whenever said first and/or second health data of a said first user and/or second user are registered.

7. Procedure according to claim 1, comprising:
acquiring at least a real ranking of a plurality of users,
comparing said real ranking with said ideal ranking relating to said same quantity of users to detect any discrepancies between said time data of said ideal ranking and said time data of said real ranking, and
correcting said time data in said database so as to eliminate said discrepancies.

8. Procedure according to claim 1, comprising:
diagnosing a real urgency index for at least one said first user;
checking, subsequent to said analysis phase, that said urgency index determined by said first algorithm corresponds to said real urgency index;
ending said test phase if said test phase result is positive;
if said test phase result is negative, revising a sequence of questions made in said interrogation phase by executing a third algorithm configured to retrace said questions addressed to said first user so as to identify a said question that led to the mismatch between said determined and real urgency indices and modifying said sequence to reduce or completely eliminate the possibility of negative outcomes of said test phase.

9. Remote management system for emergency rooms comprising means for implementing a procedure according to claim 1.

10. Computer program comprising instructions that, when the program is executed by said computer, enable the computer to execute a procedure according to claim 1.

11. Computer-readable storage medium comprising instructions that, when executed by said computer, enable the computer to execute a procedure according to claim 1.
